(19) United States
(12) Patent Application Publication
(10) Pub. No.: US 2012/0204880 A1
(43) Pub. Date: Aug. 16, 2012

(54) PATIENT INTERFACE DEVICE WITH ADJUSTABLE CHEEK SUPPORT

(75) Inventors: David W. Smith, Oakmont, PA (US); Gregory John Jablonski, Butler, PA (US); Derrick Blake Andrews, Merkleton, PA (US); Harold Allen Lockhart, Mt. Pleasant, PA (US)

(73) Assignee: KONINKLIJKE PHILIPS ELECTRONICS N.V., EINDHOVEN (NL)

(21) Appl. No.: 13/503,076

(22) PCT Filed: Sep. 28, 2010
(86) PCT No.: PCT/IB2010/054361

§ 371 Date: Apr. 20, 2012

Related U.S. Application Data
(60) Provisional application No. 61/254,269, filed on Oct. 23, 2009.

(51) Int. Cl. A61M 16/06

(52) U.S. Cl. 128/206.24

(57) ABSTRACT

A patient interface device includes a main frame having a central support portion and first and second arms extending from the central support portion. A cushion component is coupled to the central support portion. A first cheek support element is coupled to the first arm. The first cheek support element includes a first cheek support portion that engages the face and is selectively moveable relative to the first arm to adjust the linear distance between the first cheek support portion and the cushion component. A second cheek support element is coupled to the second arm. The second cheek support element includes a second cheek support portion structured to engage the patient's face and is selectively moveable relative to the second arm to adjust the linear distance between the second cheek support portion and the cushion component.

### CROSS-REFERENCE TO RELATED APPLICATIONS

This patent application claims the priority benefit under 35 U.S.C. §371 of international patent application no. PCT/IB2010/054361, filed Sep. 28, 2010, which claims the priority benefit under 35 U.S.C. §119(e) of U.S. Provisional Application No. 61/254,269 filed on Oct. 23, 2009, the contents of which are herein incorporated by reference.

### BACKGROUND OF THE INVENTION

1. Field of the Invention

The present invention relates to patient interface devices, and, in particular, to a patient interface device having a selectively adjustable cheek support element.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a cushion component on the face of a patient. The cushion component may be, without limitation, a nasal mask that covers the patient's nose, a nasal cushion having nasal prongs that are received within the patient's nares, a nasal/oral mask that covers the nose and mouth, or full face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head. Because such patient interface devices are typically worn for an extended period of time, it is important for the headgear to maintain the cushion component of the device in a tight enough seal against the patient's face without discomfort.

For patient interface devices, a key engineering challenge is to balance patient comfort against the stability of the device as well as minimize unintentional gas leakage at the patient-cushion interface. As a patient changes sleeping positions through the course of the night, the cushion components of patient interface devices may become dislodged, and the seal against the patient's face may be broken. A dislodged cushion component can be stabilized by the increasing strapping force provided by the headgear, but increased strapping force tends to reduce patient comfort. This design conflict is further complicated by the widely varying facial geometries that a given patient interface device design needs to accommodate. Such wide variation in facial geometry is demonstrated in Table 1 below (source: PeopleSize2008 database, OpenErgonomics Ltd.).

TABLE 1

| | 5% US population | 95% US population | Variation |
|---|---|---|---|
| | Min (mm) | Max (mm) | (mm) |
| Nose Tip Depth | 15 | 23 | 8 |
| Nose Length | 44 | 57 | 13 |
| Eye corner to | 151 | 184 | 33 |
| back of head | | | |
| Nose base to | 182 | 216 | 34 |
| back of head | | | |
| Cheekbone width | 126 | 150 | 24 |
| Head Breadth | 140 | 167 | 27 |

To further complicate this issue, individual tolerances for comfort also vary widely. For instance, given the same level of mechanical pressure on a specific area of the face, one person may develop significant marks, blisters, or open sores, while another person may feel no effects at all.

### SUMMARY OF THE INVENTION

In one embodiment, a patient interface device is provided that includes a main frame having a central support portion and a first arm and a second arm extending from the central support portion, a cushion component, such as a nasal cushion, a nasal mask and a nasal/oral mask, coupled to the central support portion, and a first cheek support element coupled to the first arm. The first cheek support element includes a first cheek support portion structured to engage the face of the patient and being selectively moveable relative to the first arm wherein movement of the first cheek support element adjusts a first linear distance between the first cheek support portion and the cushion component. In an exemplary embodiment, the patient interface device further includes a second cheek support element coupled to the second arm, the second cheek support element including a second cheek support portion structured to engage the face of the patient and being selectively moveable relative to the second arm wherein movement of the second cheek support element adjusts a second linear distance between the second cheek support portion and the cushion component. In this embodiment, the first cheek support element is selectively moveable along the first arm and the second cheek support element is selectively moveable along the second arm.

In one particular embodiment, the first arm includes a slot portion including a slot, wherein the first cheek support element includes a leg portion coupled to the first cheek support portion, and wherein the leg portion is received within the slot. The first cheek support portion may include a curved element extending form the leg portion, and the leg portion may be straight leg portion. Also, the leg portion may be held within the slot by a friction fit and may be slideable within the slot. The leg portion may also be held within the slot by a moveable coupling mechanism, such as such as discrete positional locating elements, push button style locking elements, or one way snaps for a fixed position and may be slideable within the slot.

In another particular embodiment, the slot portion includes a strap loop for receiving a strap of a headgear.

A method of fitting a patient interface device is also provided. The method includes receiving the patient interface device, wherein the patient interface device includes: (i) a main frame, the main frame having a central support portion and a first arm and a second arm extending from the central support portion, (ii) a cushion component coupled to the central support portion, and (iii) a first cheek support element coupled to the first arm, the first cheek support element including a first cheek support portion structured to engage a face of a patient, and adjusting a first linear distance between the first cheek support portion and the cushion component by moving the first cheek support element along the first arm. The patient interface device may also include a second cheek support element coupled to the second arm, the second cheek support element including a second cheek support portion structured to engage the face of the patient, and the method may further include adjusting a second linear distance between the second cheek support portion and the cushion component by moving the second cheek support element along the second arm. In one embodiment, the first arm includes a slot portion including a slot, the first cheek support element includes a leg portion coupled to the first cheek support portion, a the leg portion is received within the slot, and the adjusting includes sliding the leg portion within the slot.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various FIG. s. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is an isometric view of a patient interface device according to one particular embodiment of the present invention;

FIG. 2 is an isometric view of the main frame of the patient interface device of FIG. 1;

FIG. 3 is an isometric view of the cheek support element of the patient interface device of FIG. 1;

FIGS. 4 and 5 are isometric views of the patient interface device of FIG. 1 wherein the cheek support elements have been adjusted to different positions; and

FIG. 6 is an isometric view of a patient interface device according to an alternative embodiment of the present invention.

### DETAILED DESCRIPTION OF THE EXEMPLARY EMBODIMENTS

Directional phrases used herein, such as, for example and without limitation, top, bottom, left, right, upper, lower, front, back, and derivatives thereof, relate to the orientation of the elements shown in the drawings and are not limiting upon the claims unless expressly recited therein. As employed, herein, the statement that two or more parts or components are “coupled” together shall mean that the parts are joined or operate together either directly or through one or more intermediate parts or components. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

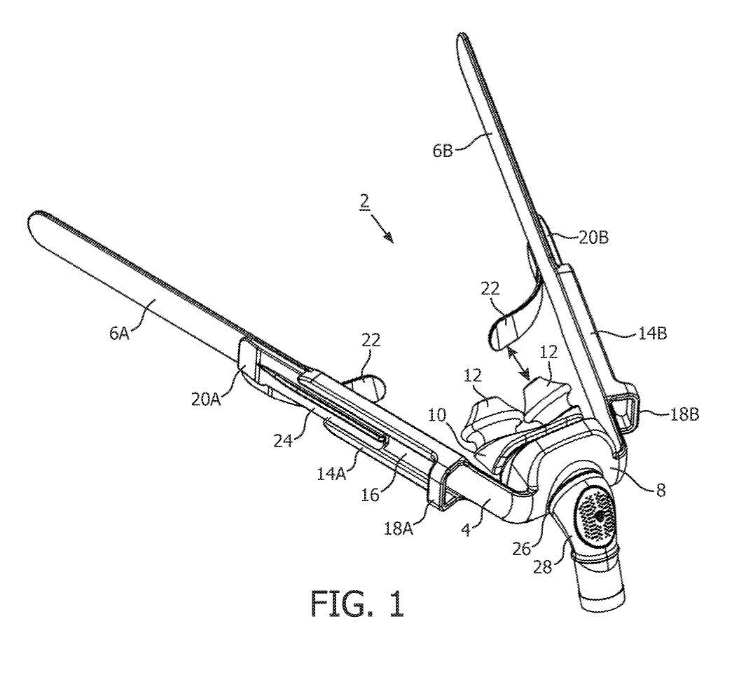

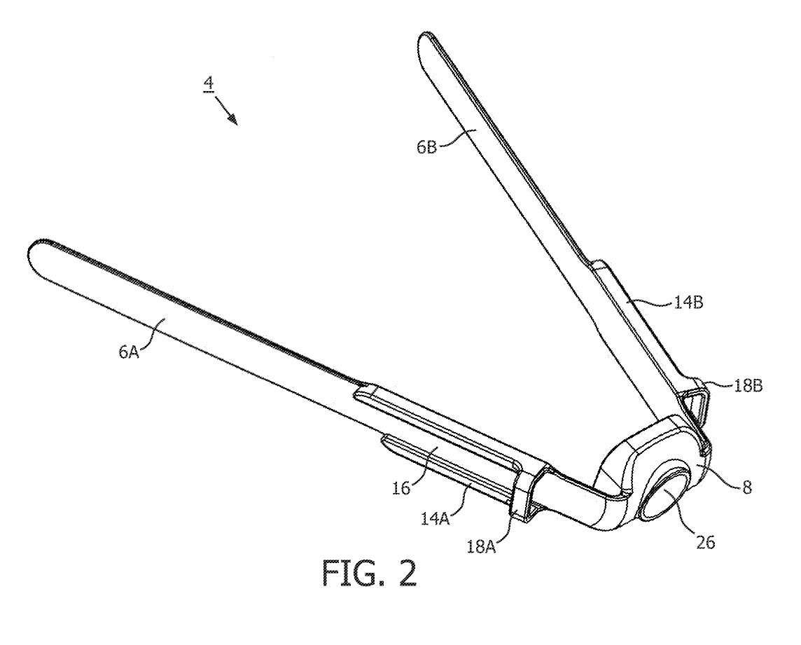

FIG. 1 is an isometric view of a patient interface device 2 according to one particular embodiment of the present invention. Patient interface device 2 includes a main frame 4, an isometric view of which is shown in FIG. 2. The present invention contemplates forming main frame 4 from a stiff material, such as a thermoplastic, and includes arms 6A and 6B and a cushion support portion 8. Arms 6A and 6B extend outwardly from cushion support portion 8 of main frame 4. Cushion support portion 8 receives and holds a nasal cushion 10 having nasal prongs 12. It is to be understood that nasal cushion 10 having individual nasal prongs 12 is but one example of a cushion component that may be used with main frame 4, and that other cushion components, such as, without limitation, a nasal/oral mask, may also be used.

Cushion support portion 8 includes an opening 26 to which there is attached a fluid coupling device 28, such as a swivel conduit as shown, for carrying fluid, such as a breathing gas, to nasal cushion 10 from an external gas source (not shown), such as a blower or other suitable device. It is to be understood that the present invention contemplates a variety of different fluid coupling devices 28 that could be attached, either permanently or selectively, to opening 26 to carry fluid to or from nasal cushion 10.

Main frame 4 also includes slot portions 14A and 14B provided on arms 6A and 6B, respectively. Each slot portion 14A, 14B includes a slot 16 therein, the purpose of which is described in below. In addition, arm 6A includes strap loop 18A and arm 6B includes strap loop 18B. Strap loops 18A and 18B are structured to receive and engage with a respective strap of a headgear (not shown) of patient interface device 2.

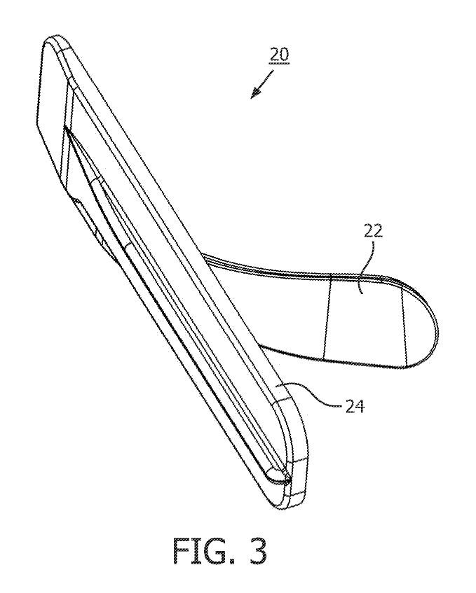

Patient interface device 2 also includes slideable cheek support elements 20A and 20B, which, in an exemplary embodiment, is also made of a stiff material, such as a thermoplastic. As seen most readily in FIG. 3, which is an isometric view of cheek support element 20, each slideable cheek support element 20A, 20B includes a cheek support portion 22 coupled to a leg portion 24. In the illustrated embodiment, cheek support portion 22 comprises a curved element extending from a straight leg portion 24. When patient interface device 2 is assembled, leg portion 24 of slideable cheek support element 20A is received in slot 16 of slot portion 14A, and leg portion 24 of slideable cheek support element 20B is received in slot 16 of slot portion 14B as shown in FIG. 1.

While the illustrated embodiment shows cheek support portion 22 as being a curved element extending from a straight leg portion 24, it is to be understood that the present invention contemplates that cheek support portion 22 can have other sizes, shapes, and configurations. For example, the patient contacting end of the cheek support portion can be enlarged or shaped to match the general contour of the region of the face that it is contacting. In addition, cheek support portion 22 can be formed from a variety of materials or combinations of materials. In an exemplary embodiment, for example, the present invention contemplates providing one or more pads (not shown) on the patient contacting side of cheek support portion 22. Such pads can be formed from any suitable material, such as cloth, foam, gel, or any other suitable material or combinations of materials. The present invention also contemplates that cheek support portion 22 can be formed from a selectively moldable material so that it can be bent, shaped or otherwise customized to suit the individual needs of the user. In which case, the cheek support portion may include an internal wire or other element that can change its shape, but retain the new shape after being so changed.

Each slideable cheek support element 20A, 20B is held in place with friction between leg 24 thereof and slot 16 of the associated slot portion 14A, 14B in a manner that permits each leg 24 to move and slide within the associated slot 16. The ability to selectively move each slideable cheek support element 20A, 20B allows the patient to adjust the linear distance, indicated by the arrow in FIG. 1, between the each slideable cheek support element 20A, 20B and nasal cushion 10. Alternatively, slideable cheek support element 20A, 20B could be moveably coupled to main frame 4 with alternative moveable coupling mechanisms such as discrete positional locating elements, push button style locking elements, or one way snaps for a fixed position.

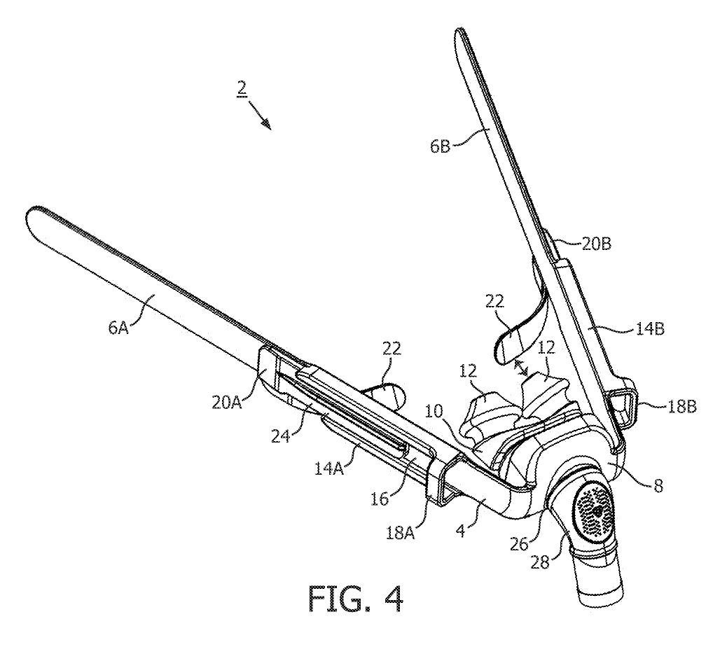

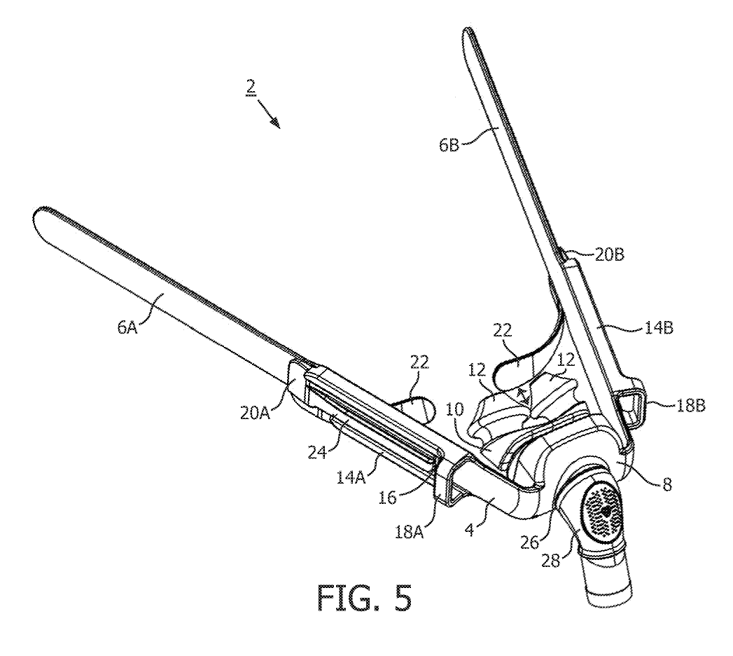

FIGS. 4 and 5 show patient interface device 2 in configurations where slideable cheek support element 20A, 20B have been adjusted to provide different linear distances between the each slideable cheek support element 20A, 20B and nasal cushion 10. This will allow the patient to find the optimal position for their particular facial anatomy and comfort level. This may be done after patient interface device 2 has been donned by the patient, or prior to patient interface device 2 being donned by the patient.

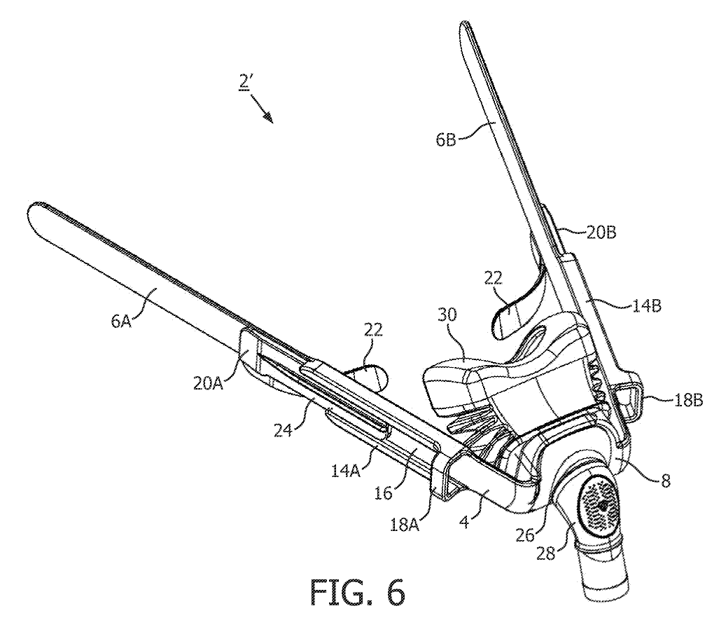

Additionally, this selective adjustability feature allows main frame 4 to be used with multiple mask/cushion styles (e.g., pillows, cradle, cone, simple). For example, FIG. 6 is an isometric view of patient interface device 2′ according to an alternative embodiment of the present invention that includes cradle style cushion 30. Various mask/cushion styles and sizes often require different distances between the patient sealing surface and the supporting frame. The selective adjustability feature provided by the cooperation between slideable cheek support element 20A, 20B and main frame 4 allows slideable cheek support element 20A, 20B to cover a large range of motion to accommodate both varying facial anatomies as well as varying mask/cushion styles.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A patient interface device, comprising:
a main frame comprising a central support portion, a first arm extending from the central support portion, and a second arm extending from the central support portion, wherein the first arm includes a straight slot portion defining a straight slot;
a cushion component coupled to the central support portion; and
a first cheek support element coupled to the first arm, the first cheek support element including a straight leg portion and a first curved cheek support portion structured to engage a face of a patient, the first curved cheek support portion having a first terminal end coupled to the straight leg portion and a second first terminal end opposite the first terminal end, the first curved cheek support portion curving continuously outwardly and away from the straight leg portion from the first terminal end to the second terminal end, wherein the straight leg portion is received within the straight slot such that the first cheek support element is selectively moveable relative to the first arm, and wherein movement of the first cheek support element adjusts a first linear distance between the first curved cheek support portion and the cushion component.

2. The patient interface device according to claim 1, further comprising a second cheek support element coupled to the second arm, the second cheek support element including a second cheek support portion structured to engage the face of the patient and being selectively moveable relative to the second arm, and wherein movement of the second cheek support element adjusts a second linear distance between the second cheek support portion and the cushion component.

3. The patient interface device according to claim 2, wherein the first cheek support element is selectively moveable along the first arm, and wherein the second cheek support element is selectively moveable along the second arm.

4. The patient interface device according to claim 1, wherein the straight leg portion is held within the straight slot by a friction fit and is slideable within the straight slot.

5. The patient interface device according to claim 1, wherein the straight leg portion is held within the straight slot by a moveable coupling mechanism and is slideable within the slot.

6. The patient interface device according to claim 1, wherein the straight slot portion includes a strap loop.

7. The patient interface device according to claim 1, wherein the cushion component is one of a nasal cushion, a nasal mask, and a nasal/oral mask.

8. A method of fitting a patient interface device, comprising:
providing a patient interface device including: (i) a main frame, the main frame comprising a central support portion (8), a first arm extending from the central support portion, and a second arm extending from the central support portion, wherein the first arm includes a straight slot portion defining a straight slot; (ii) a cushion component coupled to the central support portion; and (iii) a first cheek support element coupled to the first arm, the first cheek support element including a straight leg portion and a first curved cheek support portion structured to engage a face of a patient, the first curved cheek support portion having a first terminal end coupled to the straight leg portion and a second first terminal end opposite the first terminal end, the first curved cheek support portion curving continuously outwardly and away from the straight leg portion from the first terminal end to the second terminal end, wherein the straight leg portion is received within the straight slot such that the first cheek support element is selectively moveable relative to the first arm; and
adjusting a first linear distance between the first cheek support portion and the cushion component by sliding the straight leg portion within the straight slot.

9. The method according to claim 8, the patient interface device also includes a second cheek support element coupled to the second arm, the second cheek support element including a second cheek support portion structured to engage the face of the patient, the method further comprising adjusting a second linear distance between the second cheek support portion and the cushion component by moving the second cheek support element along the second arm.

10. The patient interface device according to claim 1, wherein the first arm includes an outer side facing away from the second arm and an inner side facing toward the second arm, and wherein the straight slot is defined on the outer side and does not extend through the first arm.

11. The patient interface device according to claim 10, wherein the straight leg portion of the first cheek support element is positioned entirely on the outer side of the first arm at least partially within the straight slot, and wherein the straight slot portion of the first arm is positioned in between the straight leg portion and the second terminal end of the first curved cheek support portion without the first cheek support element extending through the first arm from the outer side to the inner side.

12. The patient interface device according to claim 11, wherein the first curved cheek support portion is positioned entirely beneath a bottom edge of the straight slot portion of the first arm.

13. The method according to claim 8, wherein the first arm includes an outer side facing away from the second arm and an inner side facing toward the second arm, and wherein the straight slot is defined on the outer side and does not extend through the first arm.

14. The method according to claim 13, wherein the straight leg portion of the first cheek support element is positioned entirely on the outer side of the first arm at least partially within the straight slot, and wherein the straight slot portion of the first arm is positioned in between the straight leg portion and the second terminal end of the first curved cheek support portion without the first cheek support element extending through the first arm from the outer side to the inner side.

15. The method according to claim 14, wherein the first curved cheek support portion is positioned entirely beneath a bottom edge of the straight slot portion of the first arm.
